(19) United States
(12) Patent Application Publication
(10) Pub. No.: US 2025/0099289 A1
(43) Pub. Date: Mar. 27, 2025

(54) USING WICKING MATERIAL TO COLLECT LIQUID FOR TRANSPORT

(71) Applicant: PUREWICK CORPORATION, Covington, GA (US)

(72) Inventors: Raymond John Newton, Bonsall, CA (US); Joseph Mark Forehand, La Mesa, CA (US)

(21) Appl. No.: 18/974,367

(22) Filed: Dec. 9, 2024

Related U.S. Application Data
(63) Continuation of application No. 17/179,116, filed on Feb. 18, 2021, now Pat. No. 12,193,962.
Application No. 17/179,116 is a continuation of application No. 15/171,968, filed on Jun. 2, 2016, now Pat. No. 10,952,889.

(51) Int. Cl. A61F 5/44

(52) U.S. Cl. CPC A61F 5/4404; A61F 5/44; A61F 5/4401

(57) ABSTRACT

A container for collecting liquid for transport, includes a shell having a port adapted for receiving tubing and configured to define a chamber that has a window in an exterior surface of the shell. The window is so positioned that when a wicking material, which is adapted to be applied against a particular source of moisture and which is so dimensioned that when the wicking material is received within the shell and applied through the window to the particular source of moisture, the shell is at least partially closed so that when tubing is inserted through the port into the shell chamber and the shell is otherwise closed a partial vacuum can be created within the shell chamber to draw moisture into the shell chamber through the wicking material for collection as a liquid within the shell chamber for transport from the shell chamber via the tubing. A wicking material applicator includes a material having openings and formed to define an extended hollow framework about which the wicking material can be, or is, disposed.

### CROSS-REFERENCE TO RELATED APPLICATIONS

This application is a continuation of U.S. patent application Ser. No. 17/179,116 filed on 18 Feb. 2021, which is a continuation of U.S. patent application Ser. No. 15/171,968 (now issued as U.S. Pat. No. 10,952,889), filed on 2 Jun. 2016, the disclosure of each of which is incorporated herein, in its entirety, by this reference.

### BACKGROUND OF THE INVENTION

The invention generally pertains to using wicking material to collect liquid for transport and is particularly directed to a container that can be used to so collect a liquid, such as urine, from the body of a person or an animal in such a manner that the liquid can be readily transported from the container as the liquid is being collected, and is also directed to a wicking material applicator for use with such a container.

A container for collecting urine and transporting the collected urine voided from a person's body is described in U.S. Pat. No. 8,287,508 to Robert A. Sanchez. The container described in said patent is made of plastic or some other material and defines a chamber for collecting urine. The container is closed, except for having an array of openings through which urine can be drawn into the chamber for collection and at least one outlet port through which urine can be drawn away from the chamber by a transport tube inserted into the chamber. The exterior of the container is configured for enabling a moisture-wicking article to be secured over the array of openings and for enabling the secured moisture-wicking article to be disposed in contact with the region of a female body surrounding the urethral opening. A vacuum pump is attached to the transport tube in order to create a partial vacuum in the chamber in order to draw urine into the chamber for collection of the urine and in order to draw the collected urine away from the chamber.

### SUMMARY OF THE INVENTION

The invention provides a container for collecting liquid for transport, comprising: a shell having a port adapted for receiving tubing and configured to define a chamber that has a window in an exterior surface of the shell, with the window being so positioned that when a wicking material, which is adapted to be applied against a particular source of moisture and which is so dimensioned that when the wicking material is received within the shell and applied through the window to the particular source of moisture, the shell is at least partially closed so that when tubing is inserted through the port into the shell chamber and the shell is otherwise closed a partial vacuum can be created within the shell chamber to draw moisture into the shell chamber through the wicking material for collection as a liquid within the shell chamber for transport from the shell chamber via the tubing.

In one aspect of the invention, the above-described container is combined with a wicking material applicator that is adapted for so supporting the wicking material that the wicking material can be received within the shell and applied through the window against the particular source of moisture.

The invention also provides a wicking material applicator for use with a container for collecting liquid for transport that comprises: a shell having a port adapted for receiving tubing and configured to define a chamber that has a window in an exterior surface of the shell, with the window being so positioned that when a wicking material, which is adapted to be applied against a particular source of moisture and which is so dimensioned that when the wicking material is received within the shell and applied through the window to the particular source of moisture, the shell is at least partially closed so that when tubing is inserted through the port into the shell chamber and the shell is otherwise closed a partial vacuum can be created within the shell chamber to draw moisture into the shell chamber through the wicking material for collection as a liquid within the shell chamber for transport from the shell chamber via the tubing, wherein the wicking material applicator is adapted for so supporting the wicking material that the wicking material can be received within the shell and applied through the window against the particular source of moisture

The invention provides an improved container and an improved wicking material applicator that are particularly configured and adapted for applying the wicking material against a particular source of moisture.

The invention is particularly useful for persons or animals during various circumstances. These circumstances include a condition such as incontinence or a disability that limits or impairs mobility. These circumstances also include restricted travel conditions, such as sometimes experienced by pilots, drivers, workers in hazardous areas, etc. These circumstances further include collection of urine for monitoring purposes or clinical testing.

Additional features of the invention are described with reference to the detailed description.

### BRIEF DESCRIPTION OF THE DRAWING

FIG. 1 is a side view of an exemplary embodiment of a container according to the invention.

FIG. 2 is a top view of the embodiment of the container shown in FIG. 1

FIG. 3 is a view of an exemplary embodiment of a sheet of material having openings included in an exemplary embodiment of a wicking material applicator according to the invention.

FIG. 4 is a view of the exemplary embodiment of a wicking material applicator according to the invention

FIG. 5 is a view of the wicking material applicator shown in FIG. 4 with wicking material disposed about the applicator.

### DETAILED DESCRIPTION

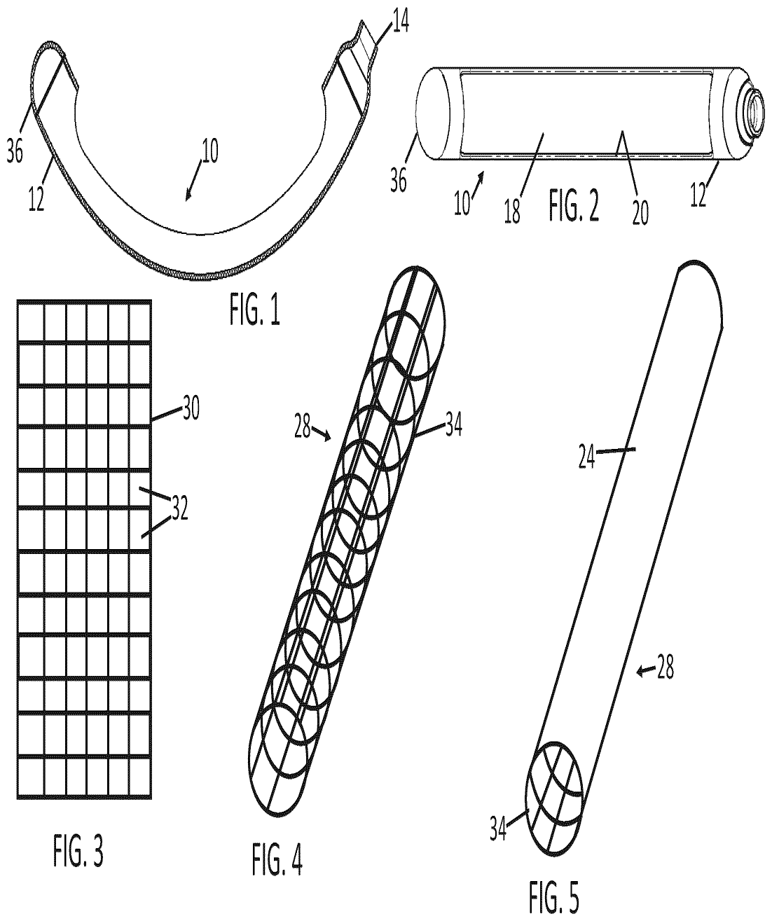

Referring to FIG. 1, an exemplary embodiment of a container 10 according to the invention includes a shell 12 having a port 14 that is adapted for receiving tubing (not shown). The shell 12 is configured to define a chamber 18 that has a window 20 in an exterior surface of the shell 12.

The window 20 is so positioned that when a wicking material 24, which is adapted to be applied against a particular source of moisture (not shown), and which is so dimensioned that when the wicking material 24 is received within the shell 12 and applied through the window 20 to the particular source of moisture, the shell 12 is at least partially closed so that when tubing is inserted through the port 14 into the shell chamber 18 and the shell 12 is otherwise closed a partial vacuum can be created within the shell chamber 18 to draw moisture into the shell chamber 18 through the wicking material 24 for collection as a liquid within the shell chamber 18 for transport from the shell chamber 18 via the tubing.

In a preferred exemplary embodiment, the shell 12 is made of a compliant impermeable plastic material, such as silicone.

In a preferred exemplary embodiment, the portion of the shell 12 adjacent the window 20 is arched so that the wicking material can be better applied against the region of a female body surrounding the urethral opening.

An exemplary embodiment of a wicking material applicator 28 is described with reference to FIGS. 3, 4 and 5. The applicator 28 includes material having symmetrical or non-symmetrical openings and formed to define an extended hollow framework about which the wicking material can be, or is, disposed. In the exemplary embodiment shown in FIG. 5, material 30, such as porous spun plastic or plastic netting material, having openings 32, as shown in FIG. 3, is formed to define an extended hollow framework 34, as shown in FIG. 4, about which framework 34 wicking material 24 can be, or is, disposed, as shown in FIG. 5. Preferably, the material 30 is flexible polypropylene. In other embodiments, the material is nylon, polyester, some other plastic or a natural material.

Because the preferred framework 34 is flexible, the wicking material applicator 28 is compliant and is dimensioned accordingly to fit snuggly within the shell chamber 18 so that the wicking material can be pressed through the window 20 against the particular source of moisture.

An exemplary method of assembling a wicking material applicator 28 within the chamber 18 of the shell includes the steps of:
(a) disposing a wicking material 24, such as tubular gauze, about a hollow plastic pipe (not shown);
(b) inserting the pipe bearing the wicking material 24 into the shell chamber 18 through the port 14 in one end of the shell whereupon the wicking material 24 is positioned so that the wicking material 24 can be applied through the window 20 against a particular source of moisture;
(c) forming a wicking material applicator 28 from material having symmetrical or non-symmetrical openings to define the extended hollow framework 34 that is shown in FIG. 4;
(d) inserting the extended hollow framework 34 through the pipe so that the framework 34 is coextensive with the wicking material 24; and
(e) removing the pipe from the shell chamber 18 while simultaneously grasping both the wicking material 24 and the extended hollow framework so that the wicking material 24 is disposed about the extended hollow framework 34 to provide an assembled a wicking material applicator 28 that is so positioned that the wicking material 24 can be applied through the window 20 against a particular source of moisture.

Alternatively, the wicking material applicator 28 is assembled outside of the shell chamber 18 and is then positioned within the shell chamber by inserting the applicator 28 through the window 20 and flexing the shell 12 around and over the sides of the wicking material applicator 28.

Preferably, the method of assembling the applicator 28 outside the shell chamber 18 includes the steps of
(a) disposing a wicking material 24, such as tubular gauze, about a hollow plastic pipe (not shown);
(b) flexing a sheet of the flexible material 30 to define the extended hollow framework 34 that is shown in FIG. 4;
(c) inserting the extended hollow framework 34 through the pipe so that the framework 34 is coextensive with the wicking material 24;
(d) removing the pipe while simultaneously grasping both the wicking material 24 and the extended hollow framework 34 so that the wicking material 24 is disposed about the extended hollow framework 34; and
(e) while the wicking material 24 is disposed about the extended hollow framework 34, cutting a section of the so-disposed wicking material 24 to the desired length to thereby provide an assembled a wicking material applicator 28.

In an alternative method of assembling the applicator 28 outside the shell chamber 18, wicking material such as rolled gauze is applied to a sheet of material having openings and attached thereto with an adhesive or by compression of a frame around the perimeter of the sheet. Assembly of the wicking material applicator is then completed by shaping the sheet having the wicking material applied thereto into an extended hollow framework and maintaining the shape of extended hollow framework with adhesive or adhesive tape. Alternatively, the shape of the extended hollow framework is maintained due to compression when inserted into the shell 12.

Different embodiments of a container for a urine collection device according to the invention are configured for use by both adult and pediatric applications, and for veterinary applications involving animals of different species and sizes.

The benefits specifically stated herein do not necessarily apply to every conceivable embodiment of the present invention. Further, such stated benefits of the present invention are only examples and should not be construed as the only benefits of the present invention.

While the above description contains much specificity, these specifics are not to be construed as limitations on the scope of the present invention, but rather as examples of the embodiments described herein. Other variations are possible and the scope of the present invention should be determined not by the embodiments described herein but rather by the claims and their legal equivalents.

What is claimed is:

1. An apparatus for collecting liquid from a female body, the apparatus comprising:
a shell having a port in a first end adapted for receiving tubing, a second end distal to the first end, a back surface extending between the first end and the second end, and a front surface distal to at least a portion of the back surface, the shell defining a chamber and having two elongated edges spaced inwardly from the front surface that at least partially define an elongated window opening in the front surface of the shell;
a wicking material applicator disposed in the chamber; and
a wicking material disposed over the wicking material applicator, the wicking material extending across the elongated window opening.

2. The apparatus of claim 1, wherein the elongated edges form an arch that extends at least partially between the first end and the second end.

3. The apparatus of claim 1, wherein the shell at least partially defines a substantially unoccupied space.

4. The apparatus of claim 3, wherein the substantially unoccupied is located in the second end.

5. The apparatus of claim 1, wherein the shell includes a compliant impermeable plastic material.

6. The apparatus of claim 1, wherein a portion of the shell extending from the elongated window opening to the first end and a portion of the shell extending from the elongated window opening to the second end exhibit a generally cylindrical shape.

7. The apparatus of claim 1, wherein the front surface of the shell is positioned to contact a region of a female body surrounding a urethral opening.

8. The apparatus of claim 1, wherein the wicking material applicator includes a porous spun plastic or plastic netting material.

9. The apparatus of claim 1, wherein the wicking material applicator defines symmetrical openings.

10. The apparatus of claim 1, wherein the wicking material applicator defines non-symmetrical openings.

11. The apparatus of claim 1, wherein the wicking material applicator is flexible.

12. The apparatus of claim 7, wherein the wicking material applicator is initially a sheet.

13. The apparatus of claim 8, wherein the wicking material applicator is configured to be rolled into a generally cylindrical shape.

14. The apparatus of claim 1, wherein a portion of the wicking material extends outwardly from the two elongated edges of the shell.

15. The apparatus of claim 12, wherein the portion of the wicking material extending outwardly from the two elongated edges of the shell exhibits a generally cylindrical shape.

16. The apparatus of claim 1, wherein the wicking material is disposed around the wicking material applicator.

17. The apparatus of claim 1, wherein the wicking material is coextensive with the wicking material applicator.

18. The apparatus of claim 1, wherein the wicking material includes gauze.

19. A system for collecting liquid from a female body, the system comprising:
an apparatus including:
a shell having a port in a first end adapted for receiving tubing, a second end distal to the first end, a back surface extending between the first end and the second end, and a front surface distal to at least a portion of the back surface, the shell defining a chamber and having two elongated edges spaced inwardly from the front surface that at least partially define an elongated window opening in the front surface of the shell;
a wicking material applicator disposed in the chamber; and
a wicking material disposed over the wicking material applicator, the wicking material extending across the elongated window opening;
a vacuum pump in fluid communication with tubing partially received in the port, the vacuum pump configured to create a partial vacuum in the chamber to draw moisture into the chamber and transport the liquid from the chamber.

20. A method for collecting liquid from a female body, the method comprising:
positioning an apparatus to contact a wicking material extending across an elongated window defined by a shell of the apparatus against a urethral opening, the apparatus including:
the shell having a port in a first end adapted for receiving tubing, a second end distal to the first end, a back surface extending between the first end and the second end, and a front surface distal to at least a portion of the back surface, the shell defining a chamber and having two elongated edges spaced inwardly from the front surface that at least partially define the elongated window opening in the front surface of the shell;
a wicking material applicator disposed in the chamber; and
the wicking material disposed over the wicking material applicator; and
receiving liquid from the urethral opening into the wicking material.
